(19) United States
(12) Patent Application Publication
(10) Pub. No.: US 2014/0287193 A1
(43) Pub. Date: Sep. 25, 2014

(54) COVERING LAYER FOR A SANITARY ARTICLE

(71) Applicants: Stephanie Lambertz, Hirschberg (DE); Peter Rutsch, Abtsteinach (DE); Michael Zaminer, Weinheim (DE); Adrian Haaser, Moerlenbach (DE)

(72) Inventors: Stephanie Lambertz, Hirschberg (DE); Peter Rutsch, Abtsteinach (DE); Michael Zaminer, Weinheim (DE); Adrian Haaser, Moerlenbach (DE)

(73) Assignee: CARL FREUDENBERG KG, Weinheim (DE)

(21) Appl. No.: 14/009,137

(22) PCT Filed: Mar. 28, 2012
(86) PCT No.: PCT/EP12/01339

§ 371 Date: Jun. 11, 2014

(30) Foreign Application Priority Data
Apr. 7, 2011 (DE) 10 2011 016 386.7

(51) Int. Cl. A61L 15/24; D04H 1/558

(52) U.S. Cl. CPC A61L 15/24; D04H 1/558
USPC 428/138; 428/131; 264/126

(57) ABSTRACT

A covering layer for a sanitary article includes a base member of non-woven material. The non-woven material is constructed from staple fibres and has a plurality of holes formed therein which have a larger diameter than pores of the non-woven material. The staple fibres have a fibre fineness which is selected from a range from greater than 1 dtex to less than or equal to 5 dtex.

### CROSS-REFERENCE TO PRIOR APPLICATIONS

This application is a U.S. National Phase application under 35 U.S.C. §371 of International Application No. PCT/EP2012/001339, filed on Mar. 28, 2012, and claims benefit to German Patent Application No. DE 10 2011 016 386.7, filed on Apr. 7, 2011. The International Application was published in German on Oct. 11, 2012, as WO 2012/136320 under PCT Article 21(2).

The invention relates to a covering layer for a sanitary article.

### BACKGROUND

From the prior art there are known covering layers of polyethylene films which have a rough and relatively even surface. The feel of these covering layers is rough and plastic-like. There is therefore a requirement for covering layers which produce a higher level of wearing comfort.

There is known from WO 2007/114742 A1 a covering layer of fleece material which has hydrophobically finished microfibres. The microfibres have a fibre fineness of 1 dtex or less and a length of approximately from 30 to 35 mm.

The microfibres are first carded and subsequently twisted together by means of water jets. Holes are formed in the covering layer by means of the water jets. However, this covering layer is not particularly stable.

Furthermore, the covering layer has no particular softness and/or volume. The wearing comfort of the known covering layer leaves room for improvement.

### SUMMARY

In an embodiment, the present invention provides a covering layer for a sanitary article including a base member of non-woven material. The non-woven material is constructed from staple fibres and has a plurality of holes formed therein which have a larger diameter than pores of the non-woven material. The staple fibres have a fibre fineness which is selected from a range from greater than 1 dtex to less than or equal to 5 dtex.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 shows a sanitary towel having a covering layer according to an embodiment of the present invention,

FIG. 2 is a schematic view of two dual-layer composite materials, which comprise a covering layer according to an embodiment of the present invention, and

FIG. 3 is a schematic view of two triple-layer composite materials, which comprise a covering layer according to an embodiment of the present invention.

### DETAILED DESCRIPTION

The present invention recognizes a need for a covering layer which is distinguished by a high level of softness, fleeciness and compressibilty.

An embodiment of the invention provides a sanitary article which has a high level of mechanical stability with a high level of softness, fleeciness and compressibility.

A covering layer according to an embodiment of the invention for a sanitary article comprises a base member of non-woven material, the non-woven material being constructed from staple fibres and there being formed in the non-woven material a plurality of holes which have a larger diameter than the pores of the non-woven material.

According to an embodiment of the invention, there is provision for the staple fibres to have a fibre fineness which is selected from the range greater than 1 dtex and less than or equal to 5 dtex.

According to an embodiment of the invention, it has surprisingly been found that relatively coarse fibres, which are specifically not constructed as microfibres, do not impair the softness and wearing comfort of the covering layer in a negative manner. It has further surprisingly been found that, in spite of the thermal solidification of the non-woven material, the softness of its surface is retained by melting of the staple fibres. The person skilled in the art would have anticipated that the melting leads to a rough surface, which reduces the wearing comfort. Although the covering layer known from WO 2007/11 47 42 A1 also has a relatively high level of wearing comfort in the dry state, owing to capillary forces produced by microfibres it develops a moisture-retaining action. Surprisingly, although the covering layer absorbs fluid quickly, it quickly transports it into a distributor layer. In this instance, the covering layer has a textile feel, is soft and does not act precisely as a plastics, sponge or synthetic material layer. Precisely as a result of the use of staple fibres of the range greater than 1 dtex and less than or equal to 5 dtex, advantageous thermal pre-fixing is possible prior to a water-jet treatment. Owing to the staple fibres, the covering layer can be thermally fixed prior to the water jet needling. A sanitary article which has a high level of mechanical stability is thereby set out.

The holes could be edged by a ring of perforations which is not hardened with respect to the remainder of the base member by means of thermal or mechanical action. By the holes being introduced by means of water-jet needling and not by means of hot air, hot needles or calenders, the rings of perforations remain soft. The covering layer retains its compressibility and fleeciness even in the region of the rings of perforations.

The staple fibres could be constructed as multi-component fibres. In this instance, it is conceivable to use island-in-the-sea fibres, core/cladding fibres or side-by-side fibres. One component can be melted on, the other component receiving the covering layer in a stable state.

Against this background, a first component of the staple fibres could have a first melting point which is greater than or equal to 150° C., a second component of the staple fibres having a second melting point which is at least 20° C. lower than the first melting point. A thermally very stable covering layer can thus be produced, it being ensured that, when the second component is melted on, the first remains substantially unimpaired.

Against this background, the staple fibres could be constructed as dual-component fibres. The cores of the dual-component fibres could comprise polyethylene terephthalate (PET) and the coatings of the dual-component fibres could comprise polypropylene (PP). Dual-component fibres of the type PET/PP are distinguished by an advantageous resilience.

The cores of the dual-component fibres could comprise polyethylene terephthalate (PET) and the coatings of the dual-component fibres could comprise polyethylene (PE). Dual-component fibres of the type PET/PE are distinguished by an advantageous level of resilience. The use of polyethylene as a coating material results in a soft feel of the non-woven material which is produced from these dual-component fibres. In order to produce the non-woven material, mixtures comprising dual-component fibres of the type PET/PP and the type PET/PE can also be used.

In particular, it is conceivable for there to be used as staple fibres dual-component fibres whose core is provided at the periphery thereof with segments which are separated from each other and which extend in the longitudinal direction of the cores. The segments are preferably constructed in a strand-like manner.

The dual-component fibres could have a core of polypropylene which is constructed as a fibre, and on the peripheral face of which segments of polyethylene are arranged. The segments can readily be melted on and may form a firmly bonded connection with other segments, which solidifies a previously laid fibre web. Polypropylene and polyethylene provide the covering surface with a hydrophobic surface.

The staple fibres could comprise a first staple fibre type and a second staple fibre type which have different melting points. A staple fibre type can be fused or melted on, the other staple fibre type receiving the covering layer in a stable state.

The covering layer could be finished at least partially in a hydrophobic, hydrophilic and/or antibacterial manner. Owing to a hydrophobic finish, water can be repelled. With a hydrophilic finish, water adheres to the covering layer. An antibacterial finish prevents the growth of bacteria. The covering layer can thus also be used for the production of a plaster or a wound dressing.

A composite could comprise a covering layer of the previously described type, and an additional layer which is constructed as a distributor layer and which can absorb fluid and can laterally distribute it, the other layer not having any holes which are greater than their pores. The fluid can thus be directed through the holes or pores of the covering layer and be absorbed in the distributor layer for lateral distribution. Such a composite material can advantageously be used in sanitary towels.

The additional layer, which is constructed as a distributor layer, could comprise multi-component fibres. One component can be melted on with the other component receiving the distributor layer in a stable state. Against this background, the staple fibres could be constructed as dual-component fibres. The distributor layer distributes a fluid in a lateral direction.

A spacing retention layer could be arranged between the covering layer and the distributor layer and has spacing-retaining walls and through-openings for the fluid. The spacing layer brings about rapid guiding of the fluid which is directed through the holes or pores of the covering layer to the distributor layer. Owing to the spacing retention layer, in a very advantageous manner fluid is prevented from being able to return to the covering layer from the distributor layer.

The spacing retention layer could be constructed as a planar layer which is harder than the covering layer. A spacing is thereby maintained between the covering layer and distributor layer even when pressure is applied to the composite material.

The through-openings could be larger than the holes of the covering layer. A very rapid fluid discharge to the distributor layer is thereby advantageously ensured. A back-up or return flow of the fluid to the covering layer is prevented.

All the layers of the composite material could be constructed as non-woven materials. The composite materials can thereby be readily laminated to each other. It is also possible to dispose of them in an environmentally friendly manner.

The covering layer described in this instance and the composite material described in this instance can advantageously be used in a sanitary towel since the covering layer ensures a high level of wearing comfort, with good fluid discharge.

A method for producing a covering layer of the type described in this instance could comprise the following steps: laying staple fibres to form a fibre web, solidifying the fibre web by means of heat application to form a non-woven material, perforating the non-woven material by means of water jets and drying or solidifying the non-woven material by means of application of heat.

The perforation can be carried out by the pre-solidified non-woven material being guided over a structured roller, from which domes protrude. Owing to the water jets, the fibres of the non-woven material are positioned around the domes so that holes are produced. When the covering layer is hydrophobically finished, the holes cause fluid to be discharged through the holes and the covering layer at the side facing the body to remain dry.

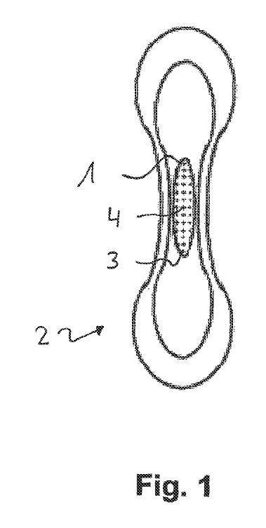

FIG. 1 shows a covering layer 1 for a sanitary article, that is to say, a sanitary towel 2 comprising a base member 3 comprising a non-woven material, the non-woven material being constructed from staple fibres and there being formed in the non-woven material a plurality of holes 4 which have a larger diameter than the pores of the non-woven material.

The staple fibres are constructed as dual-component fibres. The staple fibres are not constructed as microfibres, but instead have a fibre fineness of more than 1 dtex and less than or equal to 5 dtex.

The holes 4 are edged by a ring of perforations which is not hardened by means of thermal or mechanical action with respect to the remainder of the base member 3. The holes 4 have a hole diameter of approximately from 0.5 to 0.7 mm.

FIG. 1 shows a sanitary towel 2 having a covering layer 1 in which the dual-component fibres have a core of polypropylene which is constructed as a fibre and at the peripheral face of which segments of polyethylene are arranged. In specific terms, the covering layer has fibres of the type or brand DANAKLON.

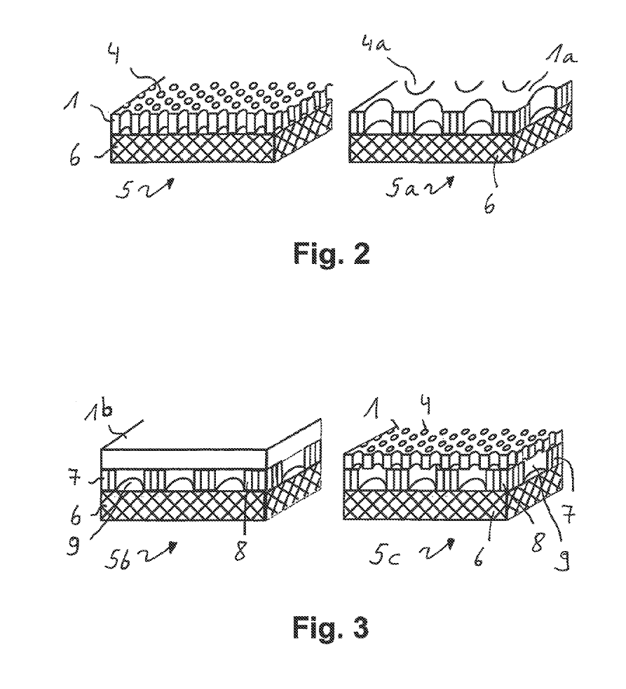

The left-hand view of FIG. 2 shows a composite material 5 comprising a covering layer 1 and another layer 6, which is constructed as a distributor layer and which can absorb fluid therein and laterally distribute it.

The right-hand view of FIG. 2 shows a composite material 5a comprising a covering layer la and another layer 6, which is constructed as a distributor layer and which can absorb fluid therein and laterally distribute it. The holes 4a of the covering layer la are larger than the holes 4 of the covering layer 1. The holes 4a have a hole diameter of approximately 2 mm.

In the case of the covering layers 1, 1a of FIG. 2, the holes 4, 4a are also each edged by a ring of perforations which is not hardened by means of thermal or mechanical action with respect to the remainder of the base member 3.

The left-hand view of FIG. 3 shows a composite material 5b, in which there is arranged between a covering layer 1b and the distributor layer a spacing retention layer 7 which has spacing-retaining walls 8 and through-openings 9 for the fluid.

The spacing retention layer 7 is constructed as a flat layer which is harder than the covering layer 1b. The spacing retention layer 7 is also harder than the distributor layer.

The right-hand view of FIG. 3 shows a composite material 5c, in which there is arranged between a covering layer 1 with holes 4 and the distributor layer a spacing retention layer 7 which has spacing-retaining walls 8 and through-openings 9 for the fluid.

The spacing retention layer 7 is constructed as a flat layer, which is harder than the covering layer 1. The spacing retention layer 7 is also harder than the distributor layer.

The covering layer 1 has a hydrophobic finish, but may also have a hydrophilic finish.

Fluid can thereby be discharged through the holes 4 and guided by the spacing retention layer 7 to the distributor layer. There, the fluid can be distributed laterally, that is to say, in a planar manner, within the distributor layer.

To this end, the through-openings 9 are larger than the holes 4 of the covering layer 1.

All the layers of the composite materials 5, 5a, 5b, 5c are constructed as non-woven materials.

The covering layers 1, 1a, 1b are produced completely from dual-component fibres.

The covering layers 1, 1a, 1b have a basis weight of from 20 to 30 g/m² and a mass of 25 g.

The dual-component fibres have a thickness of 1.4 dtex.

The holes 4, 4a are edged by a ring of perforations, which is not hardened by means of thermal or mechanical action with respect to the remainder of the base member 3, since they are introduced into the base member 3 by means of water jets.

A method for producing a covering layer 1, 1a, 1b comprises the steps of:

laying staple fibres to form a fibre web, solidifying the fibre web by means of application of heat at approximately 100° C. to form a non-woven fabric material, perforating the non-woven fabric material by means of water jets and drying or solidifying the non-woven material by means of application of heat.

In order to quantify the specific volume or fleeciness, perforated covering layers according to the invention (covering layer Erf A, B) were compared with covering layers of the same basis weight of the prior art (covering layer SdT A, B) from the company PANTEX International, Italy. The covering layers of the company PANTEX had the reference PN 053 S3 and PN 25 N 2 A 1.

The covering layer SdT A (PN 25 N 2 A1) has a hole diameter of approximately 1 mm. The covering layer SdT B (PN 053 S 3) has a hole diameter of approximately 0.5 mm.

All the covering layers examined have a basis weight of approximately 25 g/m². The covering layers of the prior art were perforated by hot needles. The covering layers according to the invention were perforated by water jets.

All the covering layers were examined with methods in accordance with the DIN standard DIN EN ISO 9073-2. The methods are set out under points 5.1 and 5.2 in the document “Test methods for nonwovens, Part 2: Determination of thickness (ISO 9073-2:1995), German version EN ISO 9073-2: 1996” of the version from February 1997.

Owing to the methods according to point 5.1 and 5.2, it was possible to establish the thicknesses of the covering layers, which are set out in the following table in mm.

The method according to point 5.1 establishes the thickness by a pressure being applied to a horizontally orientated covering layer with a vertically movable stamp. The method according to point 5.2 establishes the thickness by pressure being applied to a vertically orientated covering layer between two vertical plates.

A comparison of the thicknesses obtained by the different methods was used in order to quantify the softness or fleeciness of the covering layers.

TABLE

| Sample | Meth. Point 5.2 | Meth. Point 5.1 |
|---|---|---|
| Covering layer SdT A (1 mm) | 0.69 | 0.484 |
| Covering layer SdT B (0.5 mm) | 0.70 | 0.525 |
| Covering layer Erf B (0.5-0.7 mm) | 1.02 | 0.683 |
| Covering layer Erf A (2 mm) | 1.00 | 0.641 |

The table shows that the covering layers of the prior art having a hole diameter of approximately 1 mm have a thickness of 0.69 mm. In contrast, the covering layers according to the invention having a hole diameter of approximately 2 mm have a thickness of 1 mm. That is to say, the covering layers of the invention with relatively large holes are approximately 45% more bulky and therefore fleecier than the covering layers of the prior art with relatively large holes.

The table further shows that the covering layers of the prior art having a hole diameter of approximately 0.5 mm have a thickness of 0.7 mm. In contrast, the covering layers according to the invention having a hole diameter of approximately from 0.5 to 0.7 mm have a thickness of 1.02 mm. That is to say, the covering layers of the invention with small holes are approximately 46% more bulky and therefore fleecier than the covering layers of the prior art with small holes.

The table also shows that the covering layers of the prior art having a hole diameter of approximately 1 mm are compressed by a defined force to 70% of their initial thickness. In contrast, the covering layers according to the invention having a hole diameter of approximately 2 mm are compressed to approximately 64% of their initial thickness. That is to say, the covering layers of the invention with relatively large holes can be approximately 6% more powerfully compressed and are therefore fleecier than the covering layers of the prior art with relatively large holes.

The table finally shows that the covering layers of the prior art having a hole diameter of approximately 0.5 mm are compressed by a defined force to approximately 75% of their initial thickness. In contrast, the covering layers according to the invention having a hole diameter of approximately from 0.5 to 0.7 mm are compressed to approximately 67% of their initial thickness. That is to say, the covering layers of the invention with small holes can be approximately 8% more powerfully compressed and are therefore fleecier than the covering layers of the prior art with small holes.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1-17. (canceled)

18. A covering layer for a sanitary article comprising:
a base member of nonwoven material, the non-woven material being constructed from staple fibres and having a plurality of holes formed therein which have a larger diameter than pores of the non-woven material, wherein the staple fibres have a fibre fineness which is selected from a range from greater than 1 dtex to less than or equal to 5 dtex.

19. The covering layer according to claim 18, wherein the holes are edged by a ring of perforations which is not hardened with respect to the remainder of the base member by means of thermal or mechanical action.

20. The covering layer according to claim 18, wherein the staple fibres arc constructed as multi-component fibres.

21. The covering layer according to claim 20, wherein a first component of the staple fibres has a first melting point which is greater than or equal to 150° C. and a second component of the staple fibres has a second melting point which is at least 20° C. lower than the first melting point.

22. The covering layer according to claim 20, wherein the staple fibres are constructed as dual-component fibres.

23. The covering layer according to claim 22, wherein the dual-component fibres have a core of polypropylene which is constructed as a fibre with segments of polyethylene being arranged on a peripheral face of the core.

24. The covering layer according to claim 18, wherein the staple fibres comprise a first staple fibre type and a second staple fibre type which have different melting points from each other.

25. The covering layer according to claim 18, further comprising a finish that is at least partially hydrophobic, hydrophilic or antibacterial.

26. A composite material comprising:
a covering layer comprising a base member of non-woven material, the non-woven material being constructed from staple fibres and having a plurality of holes formed therein which have a larger diameter than pores of the non-woven material, wherein the staple fibres have a fibre fineness which is selected from a range from greater than 1 dtex to less than or equal to 5 dtex; and
an additional layer which is constructed as a distributor layer configured to absorb fluid and laterally distribute the fluid, the additional layer not having any holes which are larger than pores of the additional layer.

27. The composite material according to claim 26, wherein the additional layer comprises multi-component fibres.

28. The composite material according to claim 26, further comprising a spacing retention layer arranged between the covering layer and the distributor layer, the spacing retention layer including spacing-retaining walls and through-openings for the fluid.

29. The composite material according to claim 28, wherein the spacing retention layer is constructed as a planar layer which is harder than the covering layer.

30. The composite material according to claim 28, wherein the through-openings arc larger than the holes of the covering layer.

31. The composite material according to claim 26, wherein each of the layers is formed by non-woven materials.

32. A method for producing a covering layer for a sanitary article, comprising;
laying staple fibres to form a fibre web, wherein the staple fibres have a fibre fineness which is selected tom a range from greater than 1 dtex to less than or equal to 5 dtex;
solidifying the fibre web by means of application of heat to form a non-woven material;
perforating the non-woven material by means of water jets so as to form a plurality of holes which have a larger diameter than pores of the non-woven material; and
drying or solidifying the non-woven material by means of the application of heat.

33. A method of using the covering layer according to claim 18 comprising incorporating the covering layer into a sanitary towel.

34. A method of using the composite material according to claim 26 comprising incorporating the composite material into a sanitary towel.
